(19) United States
(12) Patent Application Publication
(10) Pub. No.: US 2026/0351386 A1
(43) Pub. Date: Nov. 5, 2026

(54) METHOD AND PROCESSING UNIT FOR PROVIDING RESULT IMAGE DATA, AND METHOD FOR PROVIDING A TRAINED DENOISING FUNCTION

(71) Applicant: Siemens Healthineers AG, Forchheim (DE)

(72) Inventors: Uwe Fabricius, Erlangen (DE); Markus Kowarschik, Erlangen (DE)

(21) Appl. No.: 19/654,793

(22) Filed: Apr. 22, 2026

(30) Foreign Application Priority Data
Apr. 30, 2025 (DE) 10 2025 116 670.6

(51) Int. Cl. G16H 30/40; G06T 5/60; G06T 5/70

(52) U.S. Cl. CPC G16H 30/40; G06T 5/60; G06T 5/70; G06T 2207/30101

(57) ABSTRACT

A computer-implemented method for providing result image data. The method includes receiving previously captured control data with vascular information relating to an examination area of an examination object, capturing image data of the examination area by a medical imaging device, generating the result image data by applying a denoising function to input data while using control data, wherein the input data is based on image data, wherein the control data specifies a plausibility condition for the structures mapped in the result image data, wherein the result image data is noise-reduced compared to the image data.

### CROSS REFERENCE TO RELATED APPLICATIONS

This application claims the benefit of DE 10 2025 116 670.6 filed on Apr. 30, 2025, which is hereby incorporated by reference in its entirety.

### FIELD

Embodiments relate to a computer-implemented method for providing result image data, a computer implemented method for providing a trained denoising function, a processing unit, a system and a computer program product.

### BACKGROUND

Interpretive image processing algorithms may be used in medical imaging for various purposes, for example for the segmentation and/or denoising of image data. In the case of segmentation, for example, endovascular objects, for example instruments and/or implants, may be detected in two-dimensional fluoroscopic images. Denoising may be used to improve image quality by reducing interference signals.

As the image quality of the input data deteriorates, for example, due to a low radiation dose in the case of X-ray images, the information content and reliability of the processed image data decrease. Structures obscured by noise may only be correctly detected and represented with limited probability. This may lead to incorrect detections and representations.

When detecting medical objects, this may mean that medical objects may be recognized in places where none actually exist or could exist. In the case of denoising algorithms, artificial structures may arise that are mistakenly reconstructed as anatomical structures or parts of medical objects.

To date, attempts have been made to optimize a balance between the sensitive detection of relevant structures and the avoidance of false detections by adapting algorithm parameters or training.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide improved mapping of an examination area of an examination object.

Embodiments are described below with regard to both methods and systems for providing result image data and methods and systems for providing a trained denoising function. Features, advantages, or alternative embodiments herein may be assigned to the other subject matter and vice versa. In other words, embodiments for providing a trained denoising function may be improved with features which are described or claimed in connection with the provision of result image data. For example, data and/or data records which are used in the methods and systems for providing result image data may have the same properties and features as the corresponding data and/or data records which are used in the methods and systems for providing a trained denoising function, and the trained denoising functions which are provided by the respective methods and systems may be used in the methods and systems for providing result image data.

A first aspect provides a computer-implemented method for providing result image data. Previously captured control data which has vascular information relating to an examination area of an examination object is received in a first step. In a further step, image data relating to the examination area is captured by a medical imaging device. In a further step, the result image data is generated by applying a denoising function to input data while using the control data. In this case, the input data is based on the image data. Moreover, the control data specifies a plausibility condition for the structures mapped in the result image data. Furthermore, the result image data is noise-reduced compared to the image data.

A computer-implemented method may be understood as a sequence of instructions executed by a computer in order to perform a specific task. The method may be written in a programming language and implemented on a computer system. In this case, the above described method may be computer-implemented at least in part, for example entirely.

The steps of the above described method may be performed at least in part simultaneously or successively, for example in the described sequence.

Receiving the previously captured control data may include reading and/or extracting data from a computer-readable data storage medium and/or receiving data from a data storage unit, for example a database. Moreover, the previously captured control data may be provided by a processing unit of one or multiple medical imaging devices, for example, the same or different imaging modalities.

The previously captured control data may be captured at an earlier point in time, for example prior to the start of the method. For example, the previously captured control data may be used as input data for the method. This previously captured control data may originate, for example, from an earlier examination or a different imaging method. The control data may include vascular information relating to an examination area of the examination object.

The examination object may be, for example, a female human patient and/or female animal patient and/or a male human patient and/or a male animal patient and/or an examination phantom.

The examination area may include a spatial area, for example a volume, of the examination object which is to be examined, for example mapped. The examination area of the examination object may include, for example, an interesting tissue and/or organ of the examination object and/or a medical object.

The vascular information may include for example 2D or 3D spatially resolved information, regarding the positioning of at least one vascular segment, for example of an artery and/or a vein, and/or a vascular malformation within the examination area of the examination object. The vascular information may advantageously include an for example 2D or 3D spatially resolved representation, for example a model and/or a mapping, of the least one vascular segment and/or the vascular malformation within the examination area of the examination object. Advantageously, the vascular information may characterize a spatial positioning, for example position and/or orientation, a size, for example a length and/or a diameter, and/or a spatial progression of the at least one vascular segment. The previously captured control data may include, for example, an especially skeletonized model, for example a central line model and/or a volume mesh model of at least one vascular segment. Alternatively or additionally, the control data may include a mapping, for example medical image data, of the at least one vascular segment.

Capturing the image data may include recording and/or receiving image data by the medical imaging device. The image data may provide a spatially resolved mapping of the examination area of the examination object, for example two-dimensional (2D) or three-dimensional (3D). For example, the image data may include 2D projection mappings of the examination area from one or multiple projection directions. These projection mappings may include, for example, X-ray images or fluoroscopic images. Alternatively or additionally, the image data may include a three-dimensional volume data record of the examination area. In addition, the image data may be temporally resolved, wherein multiple images of the examination area are captured at different points in time.

The image data may include raw data which has been provided directly by a detector, for example an X-ray detector, of the medical imaging device. Alternatively or additionally, the image data may be pre-processed at an earlier stage, for example by reconstructing raw data. Furthermore, the image data may include meta-information, for example recording parameters, patient information, procedure information and/or other relevant data.

The medical imaging device may be configured, for example, as a magnetic resonance tomography (MRT) installation and/or computed tomography (CT) installation and/or medical X-ray device and/or positron emission tomography (PET) installation and/or ultrasound device.

The input data of the denoising function may be based advantageously on the image data, for example may include the image data. Moreover, the result image data may be provided as output data of the denoising function. Providing the result image data may include storing on a computer-readable storage medium and/or displaying on a display unit and/or transmitting to a processing unit. For example, a graphic representation of the result image data may be displayed by the display unit.

The denoising function may be configured so as to remove or reduce noise in the image data. The denoising function may be configured as a classic function, for example for processing digital signals, or a trained function.

When the denoising function is configured as a classic function, the denoising function may be based, for example, on a filter method, for example a Gaussian filter, a median filter and/or a bilateral filter.

The noise in the image data may be caused, for example, by technical components of the medical imaging device and/or physical effects within the context of imaging. Alternatively or additionally, the noise in the image data may be caused by an interaction between the imaging technique, for example, X-rays and/or a magnetic field, and the examination object and/or other objects and/or electromagnetic fields. Alternatively or additionally, the noise in the image data may be caused by an physiological, movement of the examination object.

The noise and/or the noise reduction in the image data may be quantified using a metric, for example a signal-to-noise ratio (SNR) and/or a peak signal-to-noise ratio (PSNR). In addition, structural similarity indices such as a structural similarity index measure (SSIM) may be used to capture a perceived image quality more effectively than purely pixel-based metrics.

The control data specifies a plausibility condition for the structures mapped in the result image data. In this case, the plausibility condition may be understood as a rule or a criterion which is used to assess a probability and/or credibility of structures mapped in the result image data.

The structures mapped in the image data and/or result image data may include different anatomical and/or geometric features. The anatomical features may be, for example, a tissue, for example a tissue boundary, an organ, a hollow organ, for example a vascular segment, and/or an anatomical land mark, for example an ostium. In addition, the geometric features may include, for example, a contour, a contrast, an edge, a line and/or a marker object.

The vascular information included in the control data may be used to model a probability for the presence of specific structures in the image data, for example area-by-area.

For example, the plausibility condition may be introduced as an auxiliary condition in the application of the denoising function, for example an optimization process. In this case, the denoising function may be configured so as to not only reduce the noise in the image data but also to maximize the correspondence between the structures mapped in the result image data and the anatomical structures specified in the control data, for example the at least one vascular segment.

The denoising function may be configured so as to reduce the noise in the image data and simultaneously obtain relevant structures. In this case, the denoising function may use the vascular information included in the previously captured control data to assess the plausibility of structures in the result image data. Structures that are classified as unlikely according to the plausibility condition may be further denoised or removed in the result image data.

The noise-reduced result image data may have for example all the features and properties of the image data, wherein the noise in the result image data would be reduced compared to the original image data.

By using the control data when denoising the image data, the quality of the result image data may be advantageously improved. For example, a higher noise reduction may be achieved while maintaining relevant structures. This may contribute to an improved diagnostics and treatment planning.

In a further embodiment of the proposed computer-implemented method, the denoising function may be configured as a trained function. In this case, at least one parameter of the trained function may be adapted based on a comparison between training result image data and comparison image data.

The trained denoising function maps input data to output data. In so doing, For example, the output data may still depend on one or multiple parameters of the trained denoising function. The one or multiple parameters of the trained denoising function may be determined and/or adapted by training. The determination and/or adaptation of one or multiple parameters of the trained denoising function may, for example, be based on a pair of training input data and associated training output data, for example comparison output data, wherein the trained denoising function is applied to the training input data to generate training mapping data. For example, the determination and/or the adaptation may be based on a comparison between the training mapping data and the training output data, for example comparison output data. In general, a trainable denoising function, in other words a denoising function with one or multiple parameters that have not yet been adapted, is also referred to as a trained denoising function. By adapting one or multiple parameters of the trained denoising-function, for example by training the trained function, the trained denoising-function may be configured to adapt to new circumstances and to recognize and extrapolate patterns.

The trained denoising function may be adapted using supervised learning, semi-supervised learning, unsupervised learning, reinforcement learning, representation learning and/or active learning. The at least one parameter of the trained denoising function may be adapted iteratively through multiple training steps.

Other terms for trained denoising functions are trained mapping rule, trained mapping rule with trained parameters, denoising function with trained parameters, machine learning algorithm. One example of a trained denoising function is an artificial neural network, wherein the edge weights of the artificial neural network correspond to the parameters of the trained denoising function. Instead of the term “neural network”, the term “neural net” may also be used. For example, a trained denoising function may include a neural network, for example a deep artificial neural network (deep neural network, deep artificial neural network), and/or a convolutional neural network, and/or a deep convolutional network, and/or an adversarial network, and/or a deep adversarial network, and/or a generative adversarial network, a “support vector machine”, a decision tree and/or a Bayesian network. Alternatively or additionally, the trained denoising function may be based on a k-means algorithm (k-means clustering), Q-learning, genetic algorithms and/or association analysis (association rules).

Advantageously, the trained denoising function, for example the neural network, has an input layer and an output layer. In this case, the input layer may be configured to receive input data. Moreover, the output layer may be configured to provide mapping data, for example the output data. In this case, the input layer and/or the output layer may include in each case multiple pathways, for example neurons.

At least one parameter of the trained function may be adapted based on a comparison between training result image data and comparison image data. For example, the trained denoising function may be provided by an embodiment of the proposed method for providing a trained denoising function which is described in more detail below.

By adapting parameters of this function based on a comparison between training result image data and comparison image data, it is possible to achieve a precise and reliable noise-reduction of the result image data. This method may ensure an improved image quality which is of particular importance for the precise analysis and diagnosis of medical examination areas.

For example, trained denoising functions, for example neural nets, may offer the possibility of having the relationship between the location of correctly interpreted medical objects in the image data and the location of vessels in the control data be established as early as the training phase, without having to rely on model assumptions such as for example a region of influence of the vessels in the control data.

In a further embodiment of the proposed computer-implemented method, the image data may include at least one projection mapping of the examination area from a respective projection direction. The control data may include, for each of the at least one projection mapping, a respective two-dimensional vascular representation of the examination area from the projection direction of the respective projection mapping.

The at least one projection mapping may include a two-dimensional mapping of the three-dimensional examination area which is generated by projecting the three-dimensional examination area along a respective projection direction onto a two-dimensional plane. The at least one projection mapping of the examination area may include for example a 2D X-ray image or a fluoroscopic image of the examination area of the examination object. The at least one projection mapping may be captured by the medical imaging device. The medical imaging device may be configured as an X-ray device, for example a C-arc X-ray device.

In this case, the projection direction, for example angulation, may be defined as a spatial direction from which the projection mapping is recorded. The projection direction may be defined, for example by an angle of a source, for example an X-ray source, and the examination area of the examination object. For example, the image data may include multiple projection mappings which map the examination area from at least in part, for example entirely, different projection directions.

The 2D vascular representation may be configured as a planar representation of the at least one vascular segment within the examination area. Advantageously, the control data for each of the at least one projection mapping, for example for each of the multiple projection mappings, may include in each case a 2D vascular representation of the examination area, which represents, for example models and/or maps, the examination area from the respective same, for example identical, projection direction.

The 2D vascular representation may be configured as a two-dimensional mapping of the at least one vascular segment of the examination area from the same projection direction as the corresponding projection mapping. The two-dimensional vascular representation may be configured, for example, as a digital subtraction angiography (DSA). Alternatively or additionally, the 2D vascular representation may include a 2D model of the examination area. Alternatively or additionally, the 2D vascular representation may be provided by virtual projection of a 3D vascular representation along the respective projection direction of the at least one projection mapping.

The use of two-dimensional vascular representations as control data for each projection mapping may have the advantage that the plausibility condition for the structures mapped in the result image data may be precisely tailored to the respective projection direction. This may lead to an improved noise reduction and a more accurate mapping of structures in the result image data.

In a further embodiment of the proposed computer-implemented method, the control data may include a three-dimensional vascular representation of the examination area.

The 3D vascular representation may include a volumetric data record, which represents, for example models and/or maps, a spatial arrangement and structure of the at least one vascular segment within the examination area. The 3D vascular representation may have been captured, for example, using computed tomography angiography (CTA), magnetic resonance angiography (MRA), cone-beam tomography angiography or three-dimensional rotational angiography. In the case of rotational angiography, a C-arc X-ray device may be rotated about the examination object in order to capture a multiplicity of projection images from different angles, from which subsequently a three-dimensional data record may be reconstructed.

The 3D vascular representation may advantageously provide detailed spatial information regarding the progression, branches and/or diameter of the at least one vascular segment within the examination area.

Moreover, the 3D vascular representation may be used so as to generate a corresponding 2D vascular representation for each projection direction of the image data, for example of the at least one projection mapping, for example by a virtual projection along the respective projection direction. In this manner, the control data may be adapted to a geometry for capturing the image data, for example an imaging geometry of the image data.

The use of a three-dimensional vascular representation as control data may offer the technical advantage that the noise reduction and the recognition of structures in the result image data may be carried out in a more precise and more robust manner, since the spatial information regarding the vascular structures may be used more comprehensively than when using two-dimensional vascular representations.

In a further embodiment of the proposed computer-implemented method, the control data may include a vascular representation of the examination area. The control data may include a subtraction data record of the examination area. The subtraction data record may be provided based on a subtraction of a mask data record and a fill data record. The mask data record may map the examination area in a mask phase. The fill data record may map the examination area in a fill phase.

The vascular representation may include an for example 2D or 3D spatially resolved representation, for example a mapping and/or a model, of the least one vascular segment within the examination area of the examination object.

Advantageously, the mask data record and the fill data record may be recorded by the same medical imaging device.

The mask data record may map the examination area of the examination object in a mask phase. For example, the mask data record may be recorded in the mask phase. During the mask phase, essentially no contrast medium may be present in the examination area, for example in the at least one vascular segment. Moreover, the fill data record may map the examination area of the examination object in the fill phase. For example, the fill data record may be recorded in the fill phase. In this case, the mask phase and the fill phase may refer to respective, for example different, periods of time. During the fill phase, a contrast medium, particularly one that is X-ray-opaque, may be introduced into the examination area, particularly into the at least one vascular segment.

The subtraction data record may be advantageously provided as the difference between the fill data record and the mask data record, for example on a pixel-by-pixel basis. Subtracting the mask data record from the fill data record may advantageously make it possible to highlight the at least one vascular segment filled with contrast medium within the examination area of the examination object, while simultaneously suppressing background structures. The mask data record and the fill data record may be recorded for example within the context of a digital subtraction angiography (DSA).

The use of the subtraction data record as part of the control data may be advantageous since the subtraction data record may offer a clear and precise representation of vascular structures, for example of the at least one vascular segment. This may improve the accuracy and reliability of the denoising function by providing a better basis for the plausibility condition.

In a further embodiment of the proposed computer-implemented method, the plausibility condition may, based on the vascular information, specify a higher probability for the presence of structures in the vicinity of vessels within the examination area.

The plausibility condition may be configured as a rule or a criterion which defines the probability of the presence of specific structures in the result image data in dependence upon their spatial relationship, for example a spatial distance, to the vascular information included in the control data. Based on the vascular information, the plausibility condition may specify, for example provide, an, for example spatial, probability distribution and/or an, for example spatial, weighting function for a presence of structures in the vicinity of vessels in the examination area.

Advantageously, the plausibility condition may, based on the vascular information, specify a greater probability of structures in the image data which are arranged in a spatial vicinity of vessels, for example of the at least one vascular segment. For example, a stent-brace-like structure is far less likely to be found at a considerable distance from a vascular segment vessel than in the vicinity thereof. The proximity to the vessels, for example to the at least one vascular segment, may be defined as a spatial area which is located within a predetermined spatial distance from the vessels, for example the at least one vascular segment, represented in the control data, for example in the vascular information. This spatial distance may be configured, for example, as a threshold value. In this case, the denoising function may be advantageously configured so as to classify the structures in the image data which are located in spatial proximity to the vessels represented in the control data with a greater probability, for example a greater probability value and/or weight value than real structures and retain them in the result image data. Conversely, structures which are located further away from the vessels represented in the control data may be classified with a lower probability, for example a lower probability value and/or weighting value than real structures and may be possibly interpreted as noise, for example as an artifact.

The interaction between the plausibility condition and the denoising function may be configured in such a manner that the denoising function when applied to the input data, for example the image data, takes into account a spatial relationship between image points representing structures, for example structures mapped by the respective image point, to the respective next at least one vascular segment. In this case, the plausibility condition may specify the probability for the presence of structures discretely or continuously in dependence upon the respective spatial distance to the vessels in the examination area.

By using this plausibility condition, the denoising function may be configured so as to use the vascular information, for example as anatomical context information, from the control data, in order to improve a quality and reliability of the result image data, for example to distinguish between relevant noise structures and/or artificial structures. Using the additional information regarding the location of the vessels, a spatial probability distribution for the presence of medical objects, such as for example guide wires, catheters and/or stents, may be derived from the image data, thereby improving the result of the denoising function. In this manner, it is possible both to minimize artifacts resulting from incorrect interpretation outside the vessels and to improve the correct interpretation and representation of existing medical objects.

In a further embodiment of the proposed computer-implemented method, the denoising function may also be configured so as to identify a mapping of a medical object based on a structure mapped in the result image data and the plausibility condition and to provide the result image data having identification information.

The denoising function may be configured so as to perform, in addition to the noise reduction, an identification of structures in the result image data. For example, the denoising function may be configured so as to identify a mapping of a medical object in the result image data.

The medical object may be configured as an object or an apparatus which may be used for medical purposes. The medical object may include a surgical and/or diagnostic instrument, for example a catheter, guide wire and/or endoscope, and/or an implant, for example a stent.

The result image data may include multiple image points, for example pixels and/or voxels, with respective image values, for example intensity values and/or attenuation values. The mapping of the medical object may be configured as a representation of the medical object in the result image data, for example image values of image points of the result image data.

The identification of the mapping of the medical object in the result image data may include for example an identification of a corresponding structure in the result image data. The identification of the structure in the result image data may include identification, for example segmentation, of the image points of the result image data, which map the structure. The identification of the structure may be based, for example, on a comparison of the image values of the image points to a threshold value and/or a pattern recognition and/or a structure recognition. The identification of the mapping of the medical object in the result image data may include an identification, for example segmentation, of the image points in the result image data based on a structure mapped in the result image data, which map the structure, for example the medical object. The structure mapped in the result image data may include, for example, at least one geometric feature which is characteristic for the object to be medically identified.

Advantageously, the denoising function may be configured so as to identify the mapping of the medical object in addition based on the plausibility condition. The plausibility condition which may be specified by the control data may specify the probability for the presence of a mapping of the medical object at various positions in the result image data. For example, based on the vascular information, the plausibility condition may specify a greater probability for a presence of medical objects in the vicinity of vessels in the examination area.

The denoising function may be configured so as to provide the result image data having identification information. The identification information may be, for example, an annotation, for example a marking and/or highlighting, of the identified mapping of the medical object in the result image data. Alternatively or additionally, the identification information may specify coordinates and/or an area, for example relating to the result image data and/or of the examination area, in which the medical object, for example the mapping of the medical object, has been identified. The identification information may moreover include information relating to a property, for example an operating parameter and/or material parameter, and/or an identification, for example a type and/or a designation and/or category of the medical object. The identification information may include, for example, a type of medical object, its position, size, orientation and/or other relevant properties.

As a result, it is advantageously possible to improve the mapping of the examination area with a medical object.

In a further embodiment of the proposed computer-implemented method, it is possible to determine an actuality attribute of the control data. A usability of the control data may be checked in dependence upon the actuality attribute. In a positive case, the captured control data may be made available for the provision of the result image data. In a negative case, synthetic control data which has a predefined, for example spatially constant, distribution of values may be made available for the provision of the result image data, or updated control data may be captured and made available for providing the result image data.

The actuality attribute may include a parameter that specifies how up-to-date and/or valid the control data is. The actuality attribute may include, for example, a time stamp, a version number and/or a validity status.

The actuality attribute of the control data may be determined in various ways and include multiple technical aspects. The control data may include a time stamp which indicates a point in time when it was captured or last updated. A comparison of this time stamp with a current point in time of the system may be used to determine the age of the control data. Moreover, the control data may include a version number which is incremented each time the control data is updated. A comparison of this version number with a version defined as current may provide information on whether the control data is up-to-date.

Alternatively or additionally, a checksum may be calculated and stored for the control data. The checksum may be recalculated each time the control data is used and it may be compared with the stored checksum in order to identify changes in the control data. The control data may include in addition metadata which includes information regarding its validity and actuality. It is advantageously possible to monitor incidents which would require the control data to be updated, such as changes in a positioning of the examination object and/or changes in the positioning, for example a movement and/or changed angulation, of the imaging device and/or changes in the examination object, for example a medical procedure.

A usability of the control data may be checked in dependence upon the actuality attribute. Checking the usability of the control data may include comparing its actuality attribute with one or multiple predefined criteria. For example, the check may include, for example, comparing a time stamp and/or the age and/or a version of the control data with a respective threshold value, or verifying the validity status of the control data.

In a positive case, the captured control data may be made available for the provision of the result image data, for example for the application of the denoising function to the input data. “In a positive case” means here that if the usability check is successful, i.e. the control data is classified as usable, this control data will be used for further processing.

In a negative case, synthetic control data which has a predefined, particularly spatially constant, distribution of values, may be made available for the provision of the result image data. The synthetic control data may be artificially generated control data which includes a specific predefined structure and/or distribution, for example distribution of values. A spatially constant distribution of values may indicate that the synthetic control data, for example data points, for example pixels or voxels, of the synthetic control data have an identical value over the entire relevant space. The synthetic control data may be configured in such a manner that it has a neutral influence on the denoising function. In this case, the denoising function using the control data may always be created in such a manner that an absence of control data is equivalent to a provision of synthetic control data and may merely lead to a spatial uniform processing of the image data so that there are no disadvantages compared to implementations of this denoising function that do not use control data. If the control data forms an integral and essential component of the denoising function, for example in an implementation of the denoising function as a trained denoising function, for example as a neural net, then, by virtue of the aforementioned equivalence, absent control data may be represented by synthetic control data, such that this step may merely result in the loss of the beneficial effect of the control data, while the denoising function remains operational and the result image data remains usable, without the need to implement separate variants or operating modes of the denoising function for use with and without control data. This approach may render it possible for example to selectively deactivate the use of control data, for example based on a user input, if the control data is to be regarded as no longer current, but no updated control data is available yet.

Alternatively, in a negative case, updated control data may be captured and made available for the provision of the result image data, for example for applying the denoising function to the input data. The updated control data may be captured in a similar manner as the control data. Capturing the updated control data may include capturing new control data or updating the most recently captured control data in each case.

The selection between these two alternatives may be made based on different factors, such as the availability of resources for further imaging or an expected change in the examination area since the control data was last captured.

This approach ensures that the denoising function always operates using up-to-date or at least neutral control data. This may improve the quality and reliability of the result image data. For example, it may prevent outdated control data from leading to misinterpretations or artifacts in the result image data.

Advantageously, this method may render it possible to apply the denoising function in a robust and flexible manner, even when the conditions in the examination area have changed between capturing the control data and the image data. This may be particularly useful in dynamic medical scenarios, such as during an intervention, during which the vascular structure may change.

In a further embodiment of the proposed computer-implemented method, determining the actuality attribute of the control data may include receiving procedure information relating to a change in the examination area of the examination object between capturing the control data prior to the start of the method and capturing the image data.

The procedure information may include information relating to a medical procedure or intervention performed for example prior to the start of the method. The procedure information may include, for example, details relating to a type, a scope, a point in time and/or a localization of a procedure on the examination object.

Receiving the procedure information may include reading and/or extracting data from a computer-readable data storage medium and/or receiving data from a data storage unit, for example a database. Moreover, the procedure information may be provided by a processing unit of one or multiple medical imaging devices, for example, the same or different imaging modalities. Alternatively or additionally, the procedure information may be received based on a user input which has been acquired by an input unit.

The change in the examination area may be configured as a modification of the anatomical or physiological conditions within the examination area of the examination object. The change to the examination area may include, for example, an opening or closing of vessels, for example embolization or an opening of vessel occlusions, the insertion or removal of medical objects or implants, and/or changes to the tissue.

A point in time at which the acquisition of the control data takes place may be prior to the start of the method. For example, the acquisition of the control data is not part of the method. Moreover, the change in the examination area may have taken place between the acquisition of the control data and the acquisition of the image data, for example prior to the start of the method.

Determining the actuality attribute based on the procedure information may include an analysis of a type and/or a scope of the procedure performed prior to the start of the method. It is possible in this case to assess to what extent the procedure could have altered the examination area, for example the at least one vascular segment, and/or other aspects of the examination area relevant to the control data. Advantageously, it is possible to identify, for example assess, a validity of the control data based on the procedure information.

Advantageously, this approach renders it possible to dynamically adapt the method to changes in the examination area. As a result, accuracy and reliability of the result image data may be improved by ensuring that the control data used reflects the current situation in the examination area.

In a further embodiment of the proposed computer-implemented method, providing the result image data may include displaying a graphic representation of the result image data by a display unit.

The display unit may include, for example, a monitor, a display, a screen, a projector, data goggles or another apparatus for displaying the graphic representation of the result image data.

A graphic representation may be configured as a visual representation of the result image data.

Displaying the graphic representation of the result image data by the display unit may include displaying the graphic representation on a screen or a projection of the result image data on a display plane of the display unit.

Displaying the graphic representation renders it possible for a user, for example medical personnel, to visually capture and interpret the result image data. This may facilitate the diagnosis and treatment planning.

Advantageously, the graphic representation may be configured interactively. A user may, for example, zoom, rotate or highlight specific areas of the representation. This may take place by inputs at an input unit connected to the display unit.

A second aspect provides a computer-implemented method for providing a trained denoising function. In a first step, previously captured or simulated training control data is received which has vascular information relating to a training examination area of a training examination object. In a further step, previously captured or simulated training image data of the training examination area is received. In a further step, comparison image data of the training examination area is provided, wherein the comparison image data is noise-reduced and/or classified compared to the training image data. In a further step, training result image data is provided by applying the trained denoising function to input data while using the training control data, wherein the input data is based on the training image data, wherein the training control data specifies a plausibility condition for the training structures mapped in the training result image data. In a further step, at least one parameter of the trained denoising function is adapted based on a comparison of the training result image data with the comparison image data. In a further step, the trained denoising function is provided.

The advantages of the proposed computer-implemented method for providing a trained denoising function are essentially the same as those of the proposed computer-implemented method for providing result image data. The features, advantages or alternative embodiments mentioned herein may also be applied to the other claimed subject matter, and vice versa.

The training control data may have for example all the features and properties of the control data which has been described in relation to the method for providing the result image data and vice versa. The training control data may have been captured prior to the start of the method, for example by a medical imaging device, for example in a similar manner to capturing the control data. Alternatively, the training control data may be simulated.

The training examination area may be configured as a spatial area of the training examination object, which is used for training the denoising function. The training examination area and the training examination object may have for example all the features and properties of the examination area and the examination object, which have been described in relation to the method for providing result image data and vice versa.

Receiving the training control data may include reading and/or extracting data from a computer-readable data storage medium and/or receiving data from a data storage unit, for example a database. Moreover, the previously captured training control data may be provided by a processing unit of one or multiple medical imaging devices, for example, the same or different imaging modalities.

The simulation of the training control data may be based, for example, on a bio-physical model. Alternatively or additionally, it is possible by analyzing real vascular data to create statistical models which are then used to generate the synthetic training control data. Alternatively or additionally, artificial neural nets, such as generative adversarial networks or variational auto-encoders, may be trained to create realistic vascular structures. It is possible based on anatomical knowledge to define rules and/or boundary conditions which are taken into account when creating vascular structures. In addition, combinations of different methods may be used in order to combine the advantages of different approaches and create simulations as realistic as possible. Advantageously, different vascular progressions and/or vascular branches may be simulated in order to map anatomical variations. Pathological changes, such as stenoses, aneurysms and/or other vascular anomalies, may be incorporated into the simulation. The simulation may also be adapted to different demographic groups to take into account age- and gender-specific differences.

The vascular information may include for example 2D or 3D spatially resolved information, regarding the positioning of at least one vascular segment, for example of an artery and/or a vein, and/or a vascular malformation within the training examination area of the training examination object. The vascular information may advantageously include an for example 2D or 3D spatially resolved representation, specifically a model and/or a mapping, of at least one vascular segment and/or the vascular malformation within the training examination area of the training examination object. Advantageously, the vascular information may characterize a spatial positioning, for example position and/or orientation, a size, for example a length and/or a diameter, and/or a spatial progression of the at least one vascular segment.

Receiving the training image data may include reading and/or extracting data from a computer-readable data storage medium and/or receiving data from a data storage unit, for example a database. Moreover, the previously captured training image data may be provided by a processing unit of one or multiple medical imaging devices, for example, the same or different imaging modalities.

The previously captured or simulated training image data may be configured as image data which has been generated prior to the start of the method for providing a trained denoising function or may been generated by simulation. The training image data may have for example all the features and properties of the image data which has been described in relation to the method for providing the result image data and vice versa. Advantageously, the training image data may map the training examination area. The training image data may have been advantageously captured in a similar manner to the image data, for example by a medical imaging device. Alternatively, the training image data may be simulated.

The simulation of the training image data may be based on different methods and algorithms in order to generate medical training image data which is close to reality. For example, mathematical models, for example a Radon transform, may be used to generate synthetic X-ray images. Physical, for example bio-physical, simulations may simulate an interaction of an imaging method, such as X-rays, with different types of tissue. Monte Carlo methods, for example, may be used for this purpose. Statistical models may be created based on real medical image data for example, captured and/or measured data, which may then be used to generate synthetic training image data. Markov random fields or Gaussian mixture models, for example, may be used for this purpose. Alternatively or additionally, artificial neural nets, such as generative adversarial networks or variational auto-encoders, may be trained to generate training image data simulated close to reality. The simulation may advantageously take into account various factors, such as anatomical variations, pathological changes, imaging parameters and/or noise characteristics. It is possible by using these simulation methods to generate a large variety of training image data which may contribute to improving the robustness and generalizability of the trained denoising function.

The comparison image data may provide a spatially resolved mapping of the examination area, particularly in 2D or 3D. For example, the comparison image data may include 2D projection mappings of the training examination area from one or multiple projection directions. These projection mappings may include, for example, X-ray images or fluoroscopic images. Alternatively or additionally, the comparison image data may include a three-dimensional volume data record of the training examination area. In addition, the comparison image data may be temporally resolved, wherein multiple images of the training examination area are recorded at different points in time.

The comparison image data may also include raw data which has been provided directly by a detector, for example an X-ray detector, of the medical imaging device. Alternatively or additionally, the comparison image data may be pre-processed at an earlier stage, for example by reconstructing raw data. Furthermore, the comparison image data may include meta-information, for example imaging parameters, patient information, procedure information and/or other relevant data.

The comparison image data is noise-reduced and/or is classified compared to the training image data. Noise-reduced may mean that disturbing image artifacts or noise in the comparison image data have been reduced or removed compared to the training image data. Classified may mean that certain structures or objects in the comparison image data have been identified and/or labeled. The provision of the comparison image data may take place based on the training image data or based on further image data. For example, the comparison image data may have high-quality images of the training examination area with a considerably higher radiation dose or longer exposure time. These images may have an improved signal-noise ratio compared to the training image data. Alternatively or additionally, the provision of the comparison image data may include a combination, for example an average, of multiple images of the training examination area, for example by an iterative reconstruction algorithm and/or algebraic reconstruction methods and/or statistical reconstruction methods. Alternatively or additionally, the comparison image data may be provided by applying an image processing algorithm, such as a filter algorithm, for example a non-local mean value filter and/or a wavelet-based denoising method, to the training image data.

The classification of the comparison image data may include a manual annotation or an automated classification, for example application of an automated segmentation algorithm. In this case, mappings of anatomical structures, pathological changes and/or medical objects may be classified, for example annotated and/or segmented, in the comparison image data.

The training result image data is provided by applying the trained denoising function to the input data while using the training control data. In this case, the input data of the trained denoising function is based on the training image data. For example, the input data of the trained denoising function may include the training image data.

The training control data specifies a plausibility condition for the structures mapped in the training result image data. In this case, the plausibility condition may be understood as a rule or a criterion which is used to assess a probability and/or credibility of structures mapped in the training image data.

The input data of the trained denoising function may also be based on the training control data, and for example may additionally include the training control data.

When configuring the trained denoising function as a deep neural net, the training control data may be fed into different levels of the net. This may enable the trained denoising function to take plausibility information into account at different levels of abstraction. Integration may also be achieved by using conditional generative models, in which the training control data serves as a condition for generating the noise-reduced training result image data.

Moreover, at least one parameter of the trained denoising function is adapted based on a comparison of the training result image data with the comparison image data. The comparison may include, for example, a calculation of a difference and/or a measure of similarity between the training result image data and the comparison image data. The adaptation of the at least one parameter of the trained denoising function may be performed iteratively in order to improve, for example optimize, the correspondence between the training image data and the comparison image data.

The provision of the trained denoising function may include storage on a computer-readable storage medium and/or a transmission to a processing unit. The trained denoising function which is provided by this method may be used advantageously in a proposed method for providing result image data.

The described method for providing a trained denoising function may offer different technical advantages. By using training control data which includes vascular information, the denoising function may be optimized especially to the processing of medical image data with vascular structures. The specification of a plausibility condition by the training control data may contribute to the fact that the denoising function provides realistic and anatomically plausible results. By comparing the comparison image data and the iterative adaptation of the parameters, it is possible to achieve a high quality and reliability of the trained denoising function.

In general, a trained denoising function replicates cognitive functions that humans associate with other human minds. For example, the trained denoising function through training based on training data may be able to adapt to new circumstances and recognize and extrapolate patterns. Another term for “trained denoising function” may be “trained function”. In general, parameters of a denoising function may be adapted by training. For example, supervised training, semi-supervised training, unsupervised training, reinforcement learning and/or active learning may be used. Furthermore, representation learning (an alternative term is “feature learning”) may be used. For example, the parameters of the denoising function may be adapted iteratively through multiple training steps. For example, a specific cost function may be minimized within the training. For example, the backpropagation algorithm may be used within the training of a neural net. For example, a denoising function may include a neural net, a support vector machine, a decision tree and/or a Bayesian net, and/or the denoising function may be based on k-means clustering, Q-learning, genetic algorithms and/or association rules. For example, a neural net may be a deep neural net, a convolutional neural net or a convolutional deep neural net. Furthermore, a neural net may be an adversarial net, a deep adversarial net and/or a generative adversarial net.

A convolutional neural net is a type of neural net that uses a convolution operation instead of standard matrix multiplication in at least one of its layers (known as a “convolution layer”). For example, a convolutional layer may perform a scalar product of one or multiple convolutional kernels with the input data / input image of the convolutional layer, wherein the entries of the one or multiple convolutional kernels may be the parameters or weights that are adapted through training. For example, the Frobenius scalar product and the ReLU activation function may be used. A convolutional neural net may also include layers, for example pooling layers, entirely connected layers and normalizing layers.

By using convolutional neural nets, input images may be processed very efficiently, since a convolution operation based on different kernels may extract different image features, so that by adapting the weights of the convolution kernel, the relevant image features may be found during training. Furthermore, due to the weight distribution in the convolutional kernels, fewer parameters need to be trained, which prevents over-adapting in the training phase and enables faster training or more layers in the network, which improves the performance of the network.

In accordance with one aspect, the denoising function may include one or multiple residual networks (ResNet). For example, a ResNet may be an artificial neural net that includes at least one skip or jump connection which is used to skip at least one layer of the artificial neural net. For example, a ResNet may be a convolutional neural net that includes one or multiple skip connections which in each case skip one or multiple convolution layers. According to some examples, the ResNets may be represented as m-layer-ResNets, wherein m is the number of layers in the corresponding architecture and according to some examples may assume values of 34, 50, 101 or 152. According to some examples, such an m-layer-ResNet may include in each case (m-2)/2 skip connections.

A skip connection may be regarded as a bypass which directs the output of a preceding layer directly via one or multiple skipped layers to a layer following the one or multiple skipped layers. Instead of having to adapt a desired mapping directly, the skipped layers would then have to adapt a residual mapping that “compensates” for the directly forwarded output.

Adapting the residual mapping is easier to optimize computationally than the direct mapping. Furthermore, this mitigates the problem of vanishing/exploding gradients during optimization when training the denoising functions: If a skipped layer encounters such problems, its contribution may be skipped by regularizing the directly forwarded output. The use of ResNets therefore has the advantage that much deeper networks may be trained. As part of the planned provision of result image data based on image data, this has the advantage of enabling a more accurate prediction of result image data - and thus contributes to improved noise reduction.

A generative adversarial model (an acronym may be GA model) may include a generative function and a discriminative function, wherein the generative function generates synthetic data and the discriminative function distinguishes between synthetic and real data. By training the generative function and/or the discriminative function, the generative function may be configured on the one hand to generate synthetic data that is incorrectly classified as real by the discriminative function, on the other hand the discriminative function may be configured to distinguish between real data and synthetic data generated by the generative function. In terms of game theory, a generative adversarial model may be interpreted as a zero-sum game. The training of the generative function and/or the discriminative function may be based for example on the minimization of a cost function.

By using a GA model, it is possible based on a set of training data to generate synthetic data which has the same characteristics as the set of training data. Training the GA model may be based on unannotated data (unsupervised learning), so that the effort required to train a GA model is low.

For example, a recurrent denoising function may be a denoising function whose output depends not only on the input value and the parameters of the denoising function that have been adapted through the training process, but also on a hidden state vector, wherein the hidden state vector may be based on previous inputs that were used for the recurrent denoising function. For example, the recurrent denoising function may include additional storage states or additional structures that incorporate time delays or include feedback loops.

For example, the structure forming the basis of a recurrent denoising function may be a neural net which may be referred to as a recurrent neural net. Such a recurrent neural net may be described as an artificial neural net in which connections between nodes form a directed graph along a temporal sequence. For example, a recurrent neural net may be interpreted as a directed acyclic graph. For example, the recurrent neural net may be a recurrent neural net with finite pulse response or a recurrent neural net with infinite pulse response (wherein a net with finite pulse response may be rolled up and may be replaced by a strictly forward-looking neural net, and a net with infinite pulse response cannot be rolled up and not replaced by a strictly forward-looking neural network).

For example, the training of a recurrent neural network may be based on the BPTT algorithm (acronym for “backpropagation through time”), the RTRL algorithm (acronym for “real-time recurrent learning”) and/or genetic algorithms.

By using a recurrent denoising function, input data may be used which includes sequences of variable length. For example, this means that the method cannot only be used for a fixed number of input data records (and must be trained differently for any other number of input data records used as an input), but rather may be used for any number of input data records. This may mean that the entire set of training data may be used within the training irrespective of the number of input data records contained in different sequences and that training data is not reduced to training data which corresponds to a specific number of successive input data records.

A transformer network may be a neural network architecture that generally includes an encoder, a decoder, or both an encoder and a decoder. In some cases, the encoders and/or decoders may consist of multiple corresponding encoding layers and decoding layers. Sn attention mechanism may be present within each encoding and decoding layer. The attention mechanism, sometimes referred to as self-attention, may relate data elements (such as words or pixels) within a sequence of data elements to other data elements within that sequence. The self-attention mechanism enables the model, for example, to examine a group of image points within a medical image and determine the relative importance of other groups of image points within that medical image for the examined group of image points.

For example, the encoder may be configured to transform the input data into a numerical representation. The numerical representation may include one vector per input token. The encoder may be configured so as to implement an attention mechanism such that each vector of a token is influenced by the other tokens in the input. For example, the encoder may be configured such that the representations resolve to the desired output of the transformer network.

For example, the decoder may be configured to transform an input into a sequence of output tokens. For example, the decoder may be configured so as to implement a masked self-attention mechanism such that each vector of a token is only influenced by the other tokens on one side of a sequence.

Furthermore, the decoder may be autoregressive, which means that intermediate results, such as a previously predicted sequence of tokens, are fed back.

According to some examples, the input of the decoder may be based on the output of the encoder or be equivalent to the output of the encoder.

Moreover, the transformer network may include a classification module which may be configured so as to map the output of the encoder or decoder to a set of learned outputs.

According to some examples, the training of a transformer model may take place in two phases, a pre-training phase and a fine-tuning phase. In the pre-training phase, a transformer model may be trained on a large body of data in order to learn the underlying semantics of the problem. Such pre-trained transformer models are available for different languages. For certain applications described here, fine-tuning may include further training of the transformer network with medical texts with meanings annotated by experts and/or medical ontologies, such as RADLEX and/or SNOMED. With the latter, the transformer model may learn typical relationships and synonyms of medical terms for example, according to some examples.

One advantage of transformer networks may be that they are able to efficiently handle long-range dependencies in input data due to the attention mechanism. Moreover, encoders used in transformer networks may be capable of processing data in parallel, which saves computing resources during inference. Furthermore, due to autoregression, decoders of transformer networks may be able to iteratively generate a sequence of output tokens with great confidence.

The described models and training steps may be used, for example implemented, within the scope of the computer-implemented method for providing a trained denoising function. For example, the trained denoising function may be implemented as one of the described models, for example as a convolutional neural net, a ResNet or a transformer network.

A third aspect provides a processing unit. The processing (provision) unit is configured so as to perform a proposed computer-implemented method for providing result image data.

The processing unit may include a computing unit, a storage unit and/or an interface. The processing unit, for example the components of the processing unit, may be configured so as to perform the individual steps of the proposed method for providing result image data.

The interface may be configured to receive the previously captured control data and to capture the image data. The computing unit and/or the storage unit and/or the interface may be configured to provide the result image data by applying the denoising function to the input data while using the control data.

The computing unit may be configured as a processor, microprocessor, digital signal processor or an application-specific integrated circuit. The storage unit may be configured as a volatile or non-volatile memory, for example RAM, ROM, flash memory or hard disk storage. The interface may be configured as a network interface, serial interface or parallel interface.

The processing unit may be configured as a standalone device or as part of a larger system. The processing unit may be connected to further components, such as a display unit for displaying the result image data and/or an input unit for acquiring user inputs.

The advantages of the processing unit correspond essentially to the advantages of the proposed computer-implemented method for providing result image data. The processing unit may render possible an efficient and reliable processing of the image data while taking into account the control data in order to provide noise-reduced result image data. The features, advantages or alternative embodiments mentioned herein may also be applied to the other claimed subject matter, and vice versa.

A fourth aspect provides a system including a medical imaging device and a processing unit. The medical imaging device is configured so as to capture the image data and to provide it to the processing unit. The processing unit is configured so as to perform a proposed computer-implemented method for providing result image data and/or for providing a trained denoising function.

The medical imaging device may be configured, for example, as a magnetic resonance tomography (MRT) scanner and/or computed tomography (CT) installation and/or medical X-ray device and/or positron emission tomography (PET) installation and/or ultrasound device.

The medical imaging device may be configured so as to capture, for example record, image data. The medical imaging device may moreover be configured so as to provide the detected image data to the processing unit. This may include data transmission via a suitable interface, such as a wired or wireless network connection.

The advantages of the system correspond essentially to the advantages of the proposed processing unit and/or the proposed computer-implemented method for providing result image data and/or for providing a trained denoising function. The features, advantages or alternative embodiments mentioned herein may also be applied to the other claimed subject matter, and vice versa.

A training unit may be provided that is configured to perform a proposed method for providing a trained denoising function. In this case, the training unit may advantageously include a training interface, a training storage unit and/or a training computing unit. The training unit may be configured so as to perform a method for providing a trained denoising function, in that the training interface, the training storage unit and/or the training computing unit may be configured so as to perform the corresponding method steps.

Advantageously, the training interface may be configured so as to receive the previously captured or simulated training control data and the previously captured or simulated training image data. Moreover, the training computing unit and/or the training storage unit may be configured so as to provide the comparison image data, to provide the training result image data by applying the trained denoising function to the input data while using the training control data, to adapt at least one parameter of the trained denoising function based on a comparison of the training result image data with the comparison image data. Finally, the training interface may be configured so as to provide the trained denoising function.

The advantages of the training unit correspond essentially to the advantages of the proposed computer-implemented method for providing a trained denoising function. The features, advantages or alternative embodiments mentioned herein may also be applied to the other claimed subject matter, and vice versa.

A fifth aspect provides a computer program product with a computer program, which may be loaded directly into a storage medium of a computer, with program sections in order to perform all the steps of a proposed method for providing result image data and/or for providing a trained denoising function when the program sections are executed by the computer.

A computer program product may include software with a source code that still needs to be compiled and linked, or that simply needs to be interpreted, or an executable software code that only needs to be loaded into the storage medium of the computer, for example the processing unit, in order to run. The computer program product enables the computer-implemented method for providing result image data and/or for providing a trained denoising function to be performed quickly in an identically repeatable manner and robustly by a computer, for example a processing unit,. The computer program product may be configured so that it may perform the method steps by the computer, for example the processing unit.

The computer program product may be stored, for example, on a computer-readable storage medium or on a network or server, from where it may be loaded into the processor of a computer, for example a processing unit, which is directly connected to the computer, for example the processing unit, or may be configured as part of the computer, for example the processing unit. Furthermore, the control information of the computer program product may be stored on an electronically readable data carrier. The control information of the electronically readable data carrier may be configured in such a manner that when using the data carrier in a computer, for example a processing unit, it may perform a method. Examples of electronically readable data carriers may include a DVD, a magnetic tape or a USB stick, on which electronically readable control information, for example software, may be stored.

An implementation based largely on software may have the advantage that computers already in use, particularly processing units, may be easily upgraded via a software update. In addition to the computer program, such a computer program product may, where applicable, include additional elements such as documentation and/or additional components, as well as hardware components such as hardware keys (dongles, etc.) required for the use of the software.

Advantageously, the computer program product renders possible an efficient and flexible implementation of the method. The computer program product may be executed on different computer platforms, thereby enabling the method to be widely applied. In addition, the computer program product may be simply updated and expanded in order to integrate new functionalities or improvements of the method.

### BRIEF DESCRIPTION OF THE FIGURES

FIGS. 1 and 2 depict schematic representations of different embodiments of a computer-implemented method for providing result image data.

FIG. 3 depicts a schematic representation of an embodiment of a computer-implemented method for providing a trained denoising function.

FIG. 4 depicts a schematic representation of an embodiment of a processing unit.

FIG. 5 depicts a schematic representation of an embodiment of a training unit.

FIG. 6 depicts a schematic representation of an embodiment of a system.

### DETAILED DESCRIPTION

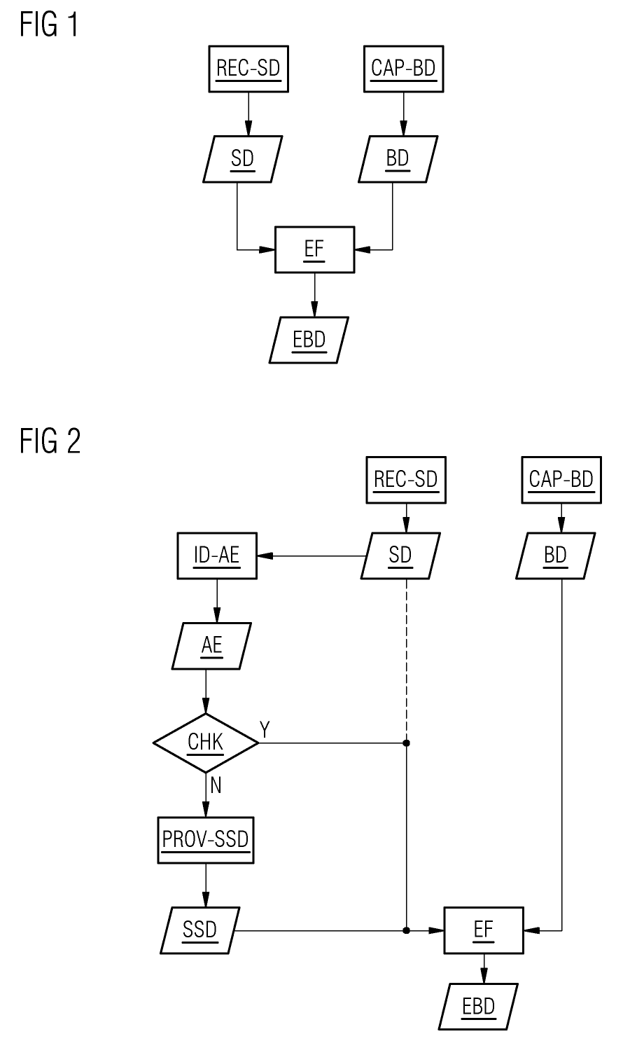

FIG. 1 depicts a schematic representation of an embodiment of a computer-implemented method for providing result image data EBD. In this case, previously captured control data SD may be received REC-SD which includes vascular information relating to an examination area of an examination object. In a further step, image data BD relating to the examination area may be captured CAP-BD by a medical imaging device. In a further step, the result image data EBD is generated by applying a denoising function EF to input data while using the control data SD. In this case, the input data may be based on the image data BD. Moreover, the control data SD may specify a plausibility condition for the structures mapped in the result image data EBD. In this case, the result image data EBD may be noise-reduced compared to the image data BD.

Advantageously, the denoising function EF may be configured as a trained function. In this case, at least one parameter of the trained function may be adapted based on a comparison between training result image data and comparison image data.

Advantageously, the image data BD may include at least one projection mapping of the examination area from a respective projection direction. In this case, the control data SD may include for each of the at least one projection mapping a respective two-dimensional vascular representation of the examination area from the projection direction of the respective projection mapping. For example, the control data SD may include a three-dimensional vascular representation of the examination area.

Alternatively or additionally, the control data SD may include a vascular representation of the examination area. In this case, the control data SD may include a subtraction data record of the examination area, which has been provided based on a subtraction of a mask data record and a fill data record. Advantageously, the mask data record may map the examination area in a mask phase. Moreover, the fill data record may map the examination area in a fill phase.

Advantageously, based on the vascular information, the plausibility condition may specify a greater probability for a presence of structures in the vicinity of vessels in the examination area.

Advantageously, the denoising function EF may moreover be configured so as to identify a mapping of a medical object based on a structure mapped in the result image data EBD and the plausibility condition and to provide the result image data having identification information.

Advantageously, providing the result image data EBD may include displaying a graphic representation of the result image data EBD by a display unit.

FIG. 2 depicts a schematic representation of a further embodiment of a computer-implemented method for providing result image data EBD. In this process, an actuality attribute AE of the control data SD may be determined ID-AE. In addition, the usability of the control data SD may be checked CHK in dependence upon the actuality attribute AE. In a positive case Y, the captured control data SD may be made available for the provision of the result image data EBD. In a negative case N, synthetic control data SSD, which has a predefined, for example spatially constant, distribution of values, may be made available for the provision of the result image data EBD. Alternatively, updated control data may be captured and made available for the provision of the result image data EBD (not illustrated here).

Advantageously, determining ID-AE the actuality attribute AE of the control data SD may include receiving procedure information relating to a change in the examination area of the examination object between the acquisition of the control data SD prior to the start of the method and the acquisition CAP-BD of the image data BD.

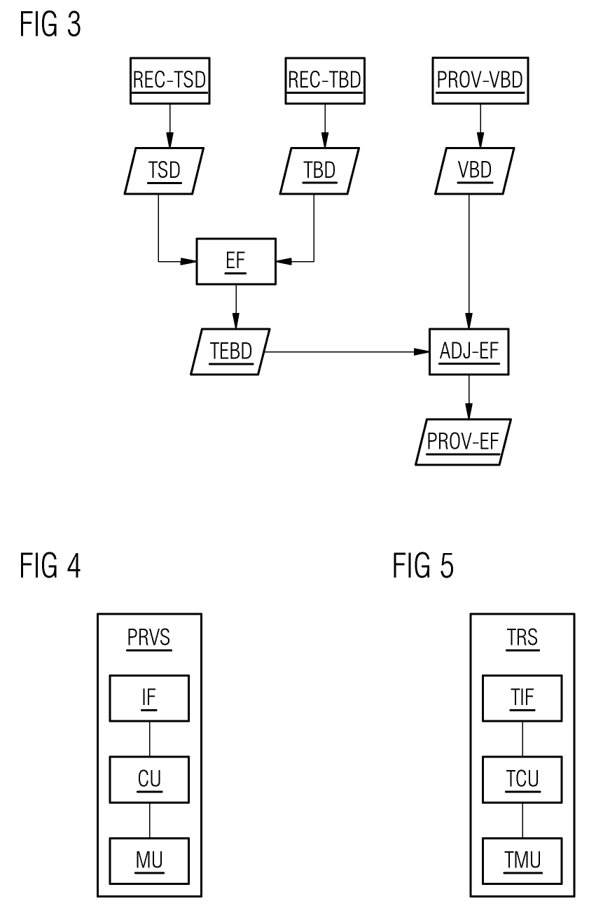

FIG. 3 shows a schematic representation of an embodiment of a computer-implemented method for providing PROV-EF a trained denoising function EF. In this case, previously captured or simulated training control data TSD may be received REC-TSD which includes vascular information relating to a training examination area of a training examination object. In a further step, previously captured or simulated training image data TBD of the training examination area may be received REC-TBD. In a further step, comparison image data VBD of the training examination area may be provided PROV-VBD. In this case, the comparison image data VBD may be noise-reduced and/or classified compared to the training image data TBD. In a further step, training result image data TEBD may be provided by applying the trained denoising function EF to input data while using the training control data TSD. In this case, the input data may be based on the training image data TBD. Moreover, the training control data TSD may specify a plausibility condition for the training structures mapped in the training result image data TEBD. In a further step, at least one parameter of the trained denoising function EF may be adapted ADJ-EF based on a comparison of the training result image data TEBD with the comparison image data VBD. In a further step, the trained denoising function may be provided PROV-EF.

FIG. 4 illustrates a schematic representation of an embodiment of a processing unit PRVS. The processing unit PRVS may include an interface IF, a computing unit CU and a storage unit MU. The processing unit PRVS, for example the components of the processing unit PRVS, may be configured so as to perform the individual steps of the method for providing result image data EBD. The processing unit PRVS may include for example a computer or may be configured as a computer.

The interface IF may be configured to receive REC-SD the previously captured control data SD and to capture CAP-BD the image data BD. The computing unit CU and/or the storage unit MU and/or the interface IF may be configured to provide the result image data EBD by applying the denoising function EF to the input data while using the control data SD.

FIG. 5 illustrates a schematic representation of an embodiment of a training unit TRS. The training unit TRS may be configured so as to perform a proposed computer-implemented method for providing PROF-EF a trained denoising function EF. In this case, the training unit TRS may advantageously include a training interface TIF, a training storage unit TMU and/or a training computing unit TCU. The training unit TRS may be configured so as to perform a method for providing PROV-EF a trained denoising function EF, in that the training interface TIF, the training storage unit TMU and/or the training computer unit TCU may be configured so as to perform the corresponding method steps.

Advantageously, the training interface TIF may be configured so as to receive REC-TSD, REC-TBD the previously captured or simulated training control data TSD and the previously captured or simulated training image data TBD. Moreover, the training computing unit TCU and/or the training storage unit TMU may be configured so as to provide PROV-VBD the comparison image data VBD, to provide the training result image data TEBD by applying the trained denoising function EF to the input data while using the training control data TSD, and to adapt ADJ-EF at least one parameter of the trained denoising function EF based on a comparison of the training result image data TEBD with the comparison image data VBD. Moreover, the training interface TIF may be configured so as to provide PROV-EF the trained denoising function EF.

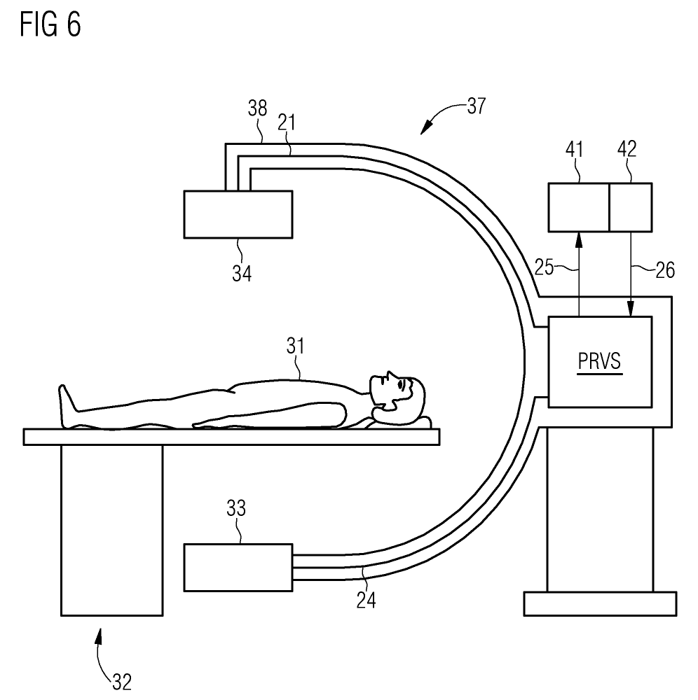

FIG. 6 illustrates a schematic representation of an embodiment of a system, including a medical imaging device, for example a C-arc X-ray device 37 and a processing unit PRVS.

The C-arc X-ray device 37 may be designed to capture image data BD of an examination object 31, for example to record projection mappings The C-arc X-ray device may include an X-ray source 33 and a detector 34, for example an X-ray detector, which are arranged opposite one another and mounted on a common C-arm 38, also referred to as a C-arc.

In order to record the medical image data BD, the arm 38 of the C-arc X-ray device 37 may be mounted so as to be movable about one or multiple axes. The system may also include a patient positioning facility 32 on which the examination object 31 is arranged. The examination object 31 may be, for example, a female human patient and/or female animal patient and/or a male human patient and/or a male animal patient and/or an examination phantom, for example a vascular phantom.

The processing unit PRVS may transmit a signal 24 to the X-ray source 33 so as to record the image data BD. The X-ray source 33 may then emit a beam of X-rays. When the beam of X-rays strikes an X-ray-sensitive layer, for example the surface, of the detector 34 following interaction with the examination object 31, the detector 34 may transmit a signal 21 to the processing unit PRVS. The processing unit PRVS may, for example, receive REC-BD the image data BD with the aid of the signal 21.

Furthermore, the C-arc X-ray device 37 may have an input unit 42, for example a keypad and a display unit 41, for example a monitor and/or a display. The input unit 42 may preferably be integrated into the display unit 41, for example in the case of a capacitive and/or resistive input display. In this case, input by a medical operator into the input unit 42 renders it possible to control the C-arc X-ray device 37. For this purpose, the input unit 42 may, for example, transmit a signal 26 to the processing unit PRVS. The display unit 41 may be advantageously configured to display a graphic representation of the result image data EBD. For this purpose, the processing unit PRVS may transmit a signal 25 to the display unit 41.

The schematic representations included in the figures described do not depict any scale or size ratios.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for providing result image data, the method comprising:
receiving previously captured control data that includes vascular information relating to an examination area of an examination object;
capturing image data of the examination area by a medical imaging device; and
generating the result image data by applying a denoising function to input data while using the previously captured control data, wherein the input data is based on the image data;
wherein the previously captured control data specifies a plausibility condition for structures mapped in the result image data;
wherein the result image data is noise-reduced compared to the image data.

2. The method of claim 1, wherein the denoising function is configured as a trained function and wherein at least one parameter of the trained function is adapted based on a comparison between training result image data and comparison image data.

3. The method of claim 1, wherein the image data comprises at least one projection mapping of the examination area from a respective projection direction and wherein the control data comprises for each of the at least one projection mapping a respective two-dimensional vascular representation of the examination area from the projection direction of the respective projection mapping.

4. The method of claim 1, wherein the control data comprises a three-dimensional vascular representation of the examination area.

5. The method of claim 1, wherein the control data comprises a vascular representation of the examination area, wherein the control data comprises a subtraction data record of the examination area, that has been provided based on a subtraction of a mask data record and a fill data record, wherein the mask data record maps the examination area in a mask phase, and wherein the fill data record maps the examination area in a fill phase.

6. The method of claim 1, wherein based on the vascular information the plausibility condition specifies a greater probability for a presence of the structures in a vicinity of vessels in the examination area.

7. The method of claim 1, wherein the denoising function is configured so as to identify a mapping of a medical object based on a structure mapped in the result image data and the plausibility condition, and to provide the result image data having identification information.

8. The method of claim 1, further comprising:
determining an actuality attribute of the control data; and
checking whether or not the control data is usable based on the actuality attribute;
wherein, in a positive case, the captured control data is made available for the provision of the result image data;
wherein, in a negative case, synthetic control data is made available for the provision of the result image data or updated control data is acquired and made available for the provision of the result image data.

9. The method of claim 8, wherein the synthetic control data includes a predefined spatially constant distribution of values.

10. The method of claim 8, wherein determining the actuality attribute of the control data comprises receiving procedure information relating to a change in the examination area of the examination object between an acquisition of the previously captured control data and an acquisition of the image data.

11. The method of claim 1, wherein providing the result image data comprises displaying a graphic representation of the result image data by a display unit.

12. A computer-implemented method for providing a trained denoising function, the method comprising:
receiving previously captured or simulated training control data that includes vascular information relating to a training examination area of a training examination object;
receiving previously captured or simulated training image data of the training examination area;
providing comparison image data of the training examination area, wherein the comparison image data is noise-reduced and/or classified compared to the training image data;
generating training result image data by applying the trained denoising function to input data while using the training control data, wherein the input data is based on the training image data, wherein the training control data specifies a plausibility condition for training structures mapped in the training result image data;
adapting at least one parameter of the trained denoising function based on a comparison of the training result image data with the comparison image data; and
providing the trained denoising function.

13. A system comprising:
a medical imaging device configured to capture image data of an examination area; and
a processing unit configured to:
receive previously captured control data that includes vascular information relating to an examination area of an examination object; and
generating result image data by applying a denoising function to input data while using the previously captured control data, wherein the input data is based on the image data, wherein the previously captured control data specifies a plausibility condition for structures mapped in the result image data, wherein the result image data is noise-reduced compared to the image data.

14. The system of claim 13, wherein the denoising function is configured as a trained function and wherein at least one parameter of the trained function is adapted based on a comparison between training result image data and comparison image data.

15. The system of claim 13, , wherein the image data comprises at least one projection mapping of the examination area from a respective projection direction and wherein the control data comprises for each of the at least one projection mapping a respective two-dimensional vascular representation of the examination area from the projection direction of the respective projection mapping.

16. The system of claim 13, wherein the control data comprises a vascular representation of the examination area, wherein the control data comprises a subtraction data record of the examination area, that has been provided based on a subtraction of a mask data record and a fill data record, wherein the mask data record maps the examination area in a mask phase, and wherein the fill data record maps the examination area in a fill phase.

17. The system of claim 13, wherein based on the vascular information the plausibility condition specifies a greater probability for a presence of structures in a vicinity of vessels in the examination area.

18. The system of claim 13, wherein the denoising function is configured so as to identify a mapping of a medical object based on a structure mapped in the result image data and the plausibility condition, and to provide the result image data having identification information.

19. The system of claim 13, wherein the processing unit if further configured to:
determine an actuality attribute of the control data; and
check whether or not the control data is usable based on the actuality attribute;
wherein, in a positive case, the captured control data is made available for provision of the result image data;
wherein, in a negative case, synthetic control data is made available for the provision of the result image data or updated control data is acquired and made available for the provision of the result image data.

20. The system of claim 19, wherein the synthetic control data includes a predefined spatially constant distribution of values.
